(19) United States
(12) Patent Application Publication
(10) Pub. No.: US 2020/0069289 A1
(43) Pub. Date: Mar. 5, 2020

(54) ULTRASOUND IMAGING USING COMPLEMENTARY CODES

(71) Applicant: The Governors of the University of Alberta, Edmonton (CA)

(72) Inventors: Roger ZEMP, Edmonton (CA); Tarek KADDOURA, Edmonton (CA); David EGOLF, Edmonton (CA)

(21) Appl. No.: 16/122,825

(22) Filed: Sep. 5, 2018

(51) Int. Cl. A61B 8/00; G01N 29/34; G01S 15/89

(52) U.S. Cl. CPC A61B 8/4483; G01S 15/8915; G01N 29/34

(57) ABSTRACT

An ultrasound imaging system for imaging a sample has an array of ultrasound transducers, a transmitter for driving the array of ultrasound transducers, a receiver that receives ultrasonic reflections from the sample, and a processor that generates an image of the sample based on a set of sub-image capture events, each sub-image capture event comprising received ultrasonic reflections. For each sub-image capture event, the transmitter transmits a sequence of transmit events from the ultrasound transducers. Each transmit event comprises a plurality of distinct waveforms directed toward separate focal zones on the sample. The sequence of transmit events comprises a sequence of distinct waveforms directed toward each focal zone. The cross-correlation level of the distinct waveforms in each transmit event is low, and the sequence of distinct waveforms is complementary.

### TECHNICAL FIELD

This relates to a system and method of ultrasound imaging of an object, and in particular, an imaging system that uses complementary codes.

### BACKGROUND

Ultrafast ultrasound imaging is providing transformational capabilities for imaging at hundreds to thousands of frames per second. Some applications include ultrafast functional brain imaging, cardiac strain imaging and shear-wave elastography. These methods also offer much larger Doppler ensemble-sizes than more conventional color- and power-Doppler methods for each pixel in an image, thus providing high sensitivity to subtle blood flow.

Current approaches for ultrafast imaging use unfocused plane wave or diverging wave transmissions. Since the transmitted energy remains distributed in a broad area (plane waves) or spreads out (diverging waves) in these cases, these approaches insonify a broad region with low levels of ultrasonic energy on each transmission. Consequently, to obtain a high quality image, several transmissions are typically required. In one example, it was found that plane wave compounding with about 1/10 of the number of transmits needed for conventional walking aperture imaging achieves image quality comparable to walking aperture. The image quality may be furthered improved through the simultaneous transmission of plane waves encoded using a Hadamard matrix, allowing for the simultaneous transmission of N plane waves in N transmits, so that additional insonification of the medium is achieved on each transmission without a reduction in framerate.

High image quality with plane-wave imaging or diverging wave imaging generally requires coherency over multiple transmit events. When tissue motion is substantial, as is the case in cardiac imaging and other applications, coherent compounding approaches suffer from severe motion artifacts. To reduce these artifacts one may use fewer transmits but at the cost of increased clutter and reduced signal-to-noise. Alternatively, to achieve high image quality at accelerated frame-rates, others have proposed multi-line imaging. However, crosstalk from multiple simultaneous transmit focal zones may limit image quality. Others have proposed Hadamard coded aperture multi-line methods, which generally require coherency over multiple transmit events.

Alternatively, focused ultrasound imaging strategies are capable of delivering greater energy to a point of interest than an unfocused ultrasound imaging modality. This gives focused modalities a fundamental signal to noise (SNR) advantage over unfocused modalities. However, in order to generate a high SNR image, a focused modality must transmit a focused beam to each area of interest. For example, if 128 lines are to be formed, and a particular depth is of interest in all of them, then the most straightforward focused imaging strategy (called “walking aperture”) requires 128 separate transmit events, each one focused at one point of interest. By contrast, unfocused plane wave imaging is capable of forming an image of the same region with as little as a single transmit.

### SUMMARY

There is provided a system and method that uses complementary arbitrary-level codes to increase the number of simultaneously transmitted focal zones. The system and method may require coherency over two or more transmit events, while offering relatively high image quality. The proposed arbitrary-level codes may offer a larger optimization space compared to more traditional binary codes, which enables both complementarity and low cross-correlation properties to reduce crosstalk. The number of parallel focal zones may be increased compared to previous multi-line methods, while still maintaining relatively high image quality. The proposed focusing may enable higher signal-to-noise ratio compared to plane-wave approaches for the same transmit energy.

According to an aspect, there is provided a system and method for ultrasound imaging using multiple parallel focal zones wherein novel code sequences are sent to each focal region. Complementary codes, such as Complementary Pseudo-Random (CPR) code pairs are introduced, such as those that possess the unique features of delta-function autocorrelation sums and low mutual cross-correlation. The design flexibility of these code pairs is used to reduce cross-correlation.

According to an aspect, there is provided a fast ultrasound imaging system comprising an array of ultrasound transducer elements, each connected to pulsing electronics with arbitrary waveform transmission capabilities, as well as connected to receiving electronics; the pulsing electronics configured to transmit to a multiplicity of focal zones within each transmit event, the focusing accomplished using transmit-delays of the transmitted waveform for each focal zone; the transmitted waveforms are selected in pairs from a set of optimized complementary pseudorandom (CPR) codes to transmit a sequential pair of transmit events; the CPR code pairs designed as sets of two sequences of numbers such that the sum of the aperiodic autocorrelations of codes in the pair is a discrete delta function or close to it; and such that the sum of aperiodic cross correlation between pairs of two sequences from different pairs is lower than a specific threshold for each lag; the receive electronics configured to receive ultrasonic echoes from each element in parallel and convert these signals to digital form; moreover, a processor to cross-correlate and filter received echoes with sequences of choice and add pairs of cross-correlated echo signals; a beamforming processor to reconstruct ultrasound scanlines for each focal zone transmitted for each transmit event pair.

According to an aspect, there is provided an algorithm for creating optimized CPR code pairs having (i) seed vector of length N (ii) computing an objective function to minimize, the objective function consisting of two terms, the first representing the normalized deviation of code pair fired at location k from being complementary, the second term representing cross-correlation interference between simultaneous beams; the minimization algorithm producing outputs of optimized CPR code pairs.

According to an aspect, there is provided an ultrasound imaging system for imaging a sample, comprising an array of ultrasound transducers, a transmitter for driving the array of ultrasound transducers, a receiver that receives ultrasonic reflections from the sample, a processor that generates an image of the sample based on a set of sub-image capture events, and a controller. Each sub-image capture event comprises received ultrasonic reflections. The controller comprises instructions to, for each sub-image capture event, cause the transmitter to transmit a sequence of transmit events from the ultrasound transducers, each transmit event comprising a plurality of distinct waveforms directed toward separate focal zones on the sample, and the sequence of transmit events comprising a sequence of distinct waveforms directed toward each focal zone, wherein a cross-correlation level of the distinct waveforms in each transmit event is below a predetermined threshold, and wherein each sequence of distinct waveforms directed toward each focal zone are complementary.

According to other aspects, the system may comprise one or more of the following features, alone or in combination: the predetermined threshold may be selected to produce a desired image quality; the sequence of distinct waveforms may be generated using nonlinear optimization algorithms; the sequence of distinct waveforms may be pseudorandom codes or Golay codes; the plurality of distinct waveforms in each transmit event may be transmitted simultaneously, and are directed toward separate focal zone using transmit delays across the array of ultrasound transducers; and the complementarity of the sequence of distinct waveforms may be such that a sum of an aperiodic autocorrelation of the sequence of distinct waveforms approximates a discrete delta function.

According to another aspect, there is provided a method of ultrasound imaging of a sample, comprising the steps of: driving an array of ultrasound transducers to transmit transmit events toward the sample; receiving ultrasonic reflections from the sample as a set of sub-image capture events, each sub-image capture event comprising a sequence of transmit events; and generating an image of the sample based on the set of sub-image capture events; wherein each transmit event comprises a plurality of distinct waveforms directed toward separate focal zones on the sample, and each sequence of transmit events comprising a sequence of distinct waveforms directed toward each focal zone, wherein a cross-correlation level of the distinct waveforms in each transmit event is below a predetermined threshold, and wherein each sequence of distinct waveforms directed toward each focal zone are complementary.

According to other aspects, the method may further comprise one or more of the following features, alone or in combination: the predetermined threshold may be selected to produce a desired image quality; the distinct waveforms may be generated using nonlinear optimization algorithms; the sequence of distinct waveforms may be pseudorandom codes or Golay codes; the plurality of distinct waveforms in each transmit event may be transmitted simultaneously, and are directed toward separate focal zone using transmit delays across the array of ultrasound transducers; and the complementarity of the sequence of distinct waveforms may be such that a sum of an aperiodic autocorrelation of the sequence of distinct waveforms approximates a discrete delta function.

In other aspects, the features described above may be combined together in any reasonable combination as will be recognized by those skilled in the art.

### BRIEF DESCRIPTION OF THE DRAWINGS

These and other features will become more apparent from the following description in which reference is made to the appended drawings, the drawings are for the purpose of illustration only and are not intended to be in any way limiting, wherein:

FIG. 1 is an example of an ultrasound imaging system.

FIGS. 2a and 2b depicts a pair of complementary codes.

FIG. 2c depicts a transmission scheme with K pairs of transmit events, with four focal zones in each transmit events, which allows 12 focal zones to be imaged.

FIG. 3 depicts an imaging scheme involving code pairs transmission, channel data decoding, line-set beamforming, and the combining of beamformed line-sets to form an image.

FIG. 4 is a graph depicting the increase in clutter introduced by simultaneous transmission with the ISL metric.

FIG. 5a-5d are graphs depicting an example of a CPR code with the tri-state version, a comparison of the output waveforms associated with transmitting an arbitrary level CPR code, a tri-state-level code, and the associated error, as well as a depiction of how the code fits within the bandwidth of the transducer.

FIG. 6a-6f depicts 3×3 point scatterer simulations comparing various imaging approaches.

FIG. 7a-7f depicts images of simulations using based in different variables.

FIG. 8a-8c are graphs showing the signal to noise ratio (SNR), contrast-to-noise ratio (CNR), and contrast-to-speckle ratio (CSR) simulation comparisons for coherently compounded plane wave imaging and complementary pseudo-random encoding scheme.

FIG. 9a-9f depicts simulated images of an axially moving grid of nine points under various conditions.

FIG. 10a-10f depicts experimentally obtained images of a sample under various conditions.

FIG. 11a-11c are graphs showing the SNR, CNR, and CSR experimental comparisons for different encoding schemes.

### DETAILED DESCRIPTION OF PREFERRED EMBODIMENTS

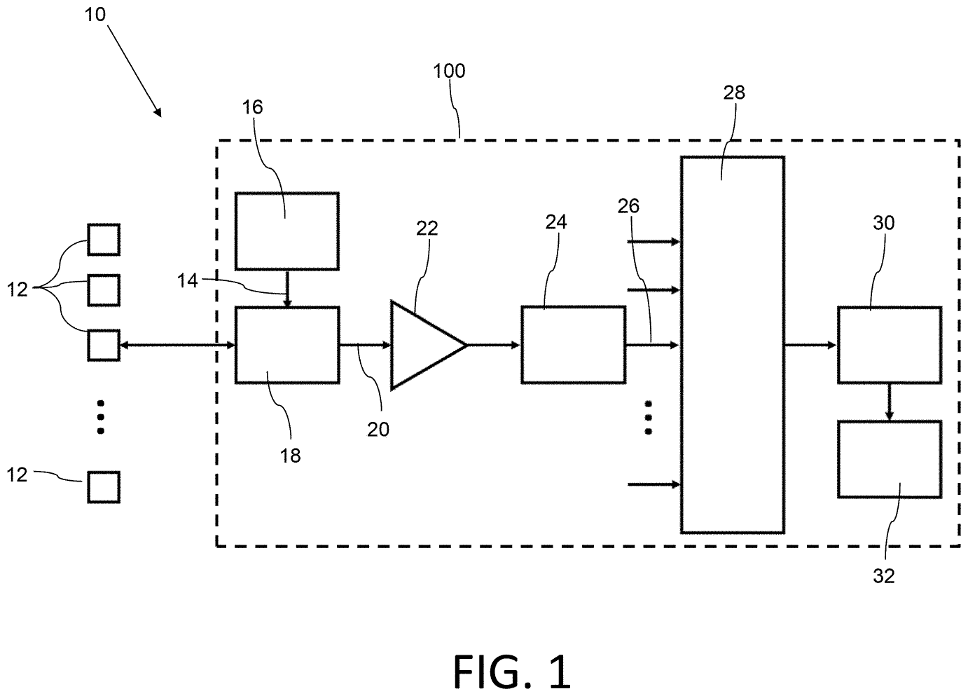

An ultrasound imaging system, generally identified by reference numeral 10, will now be described with reference to FIG. 1 through 11.

Referring to FIG. 1, there is shown an example of ultrasound imaging system 10 that may be used to implement the method described herein. It will be recognized that other designs may also be used based on the required components as described herein. In the depicted example, system 10 has an array of transducers 12 and a controller 100. Controller 100 produces an encoded transmission electronic waveform 14 from a transmission waveform generator 16 that is coupled into transducer 12 via coupler 18. Different transducers 12 may simultaneously produce transmission waveforms with different encoding. Transducers 12 receive electronic waveform 14 and produce acoustic waves. Transducer 12 also receives reflected acoustic waves and produces a received electronic waveform 20 which is routed into an amplifier 22 by coupler 18. The analogue received waveform 20 is then converted into a digital received waveform 26 by an analogue to digital converter 24. Digital waveform 26 may be stored in a memory unit 28. A processor 30 receives digital waveforms 26, which may originate from a plurality of transducers 12, and decodes digital waveforms 26 to produce an image on a display 32.

The system and method described herein use multiple simultaneous transmissions, which can be decoded to recover images approaching those acquired with serial rather than parallel transmissions.

Code-division multiple-access (CDMA) strategies have been investigated for many years in the telecommunications sector. Some of these approaches are the reason multiple cell-phone users can communicate with minimal interference. These strategies may be difficult to employ in ultrasound imaging because of the stringent image quality requirements and greater than 50 dB dynamic range expected in ultrasound images, and because scattering path-lengths are often random.

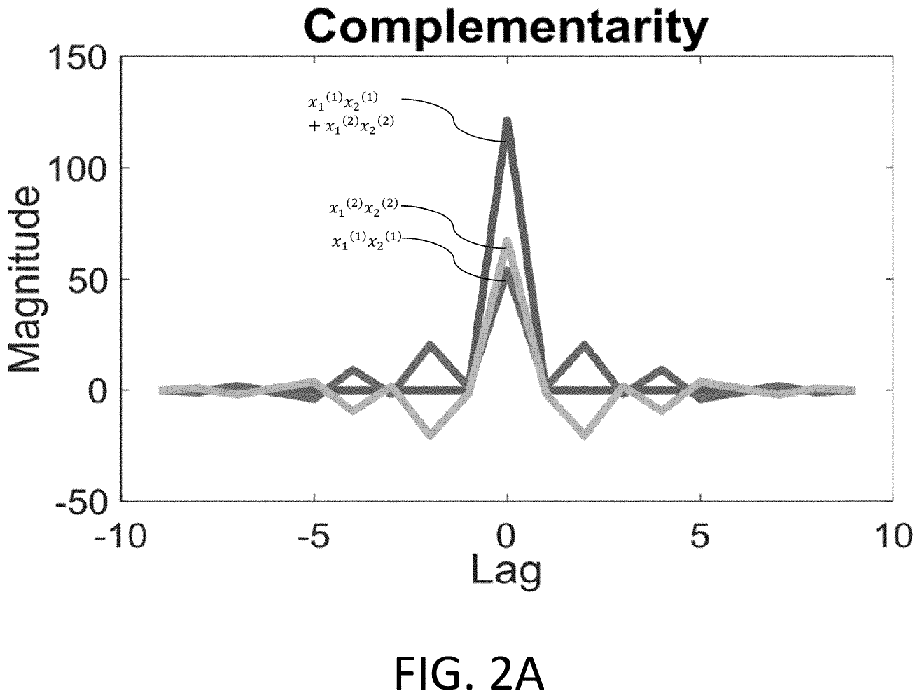

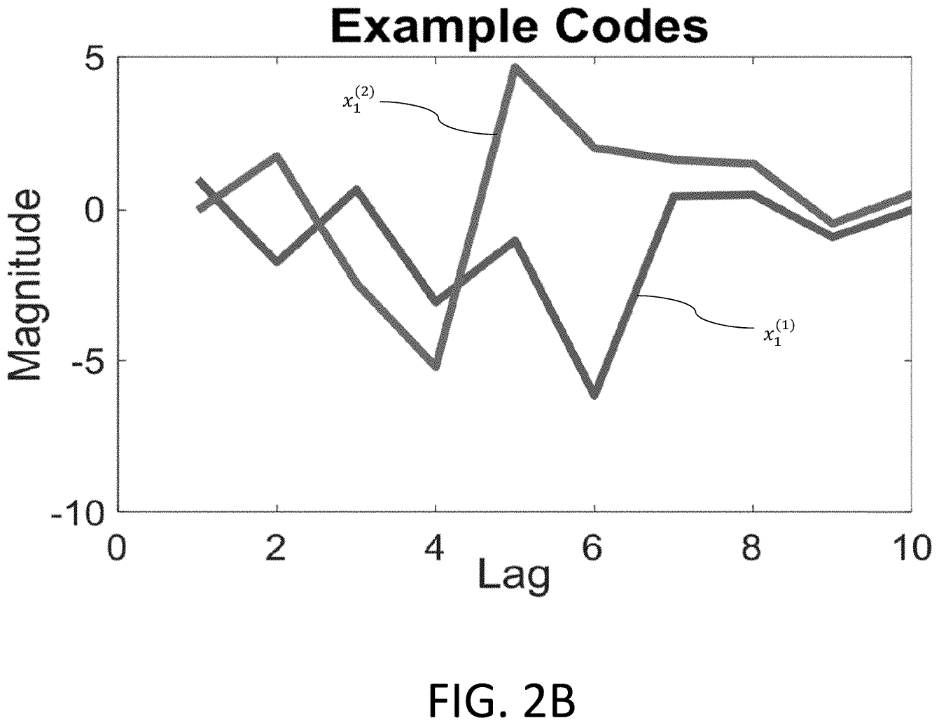

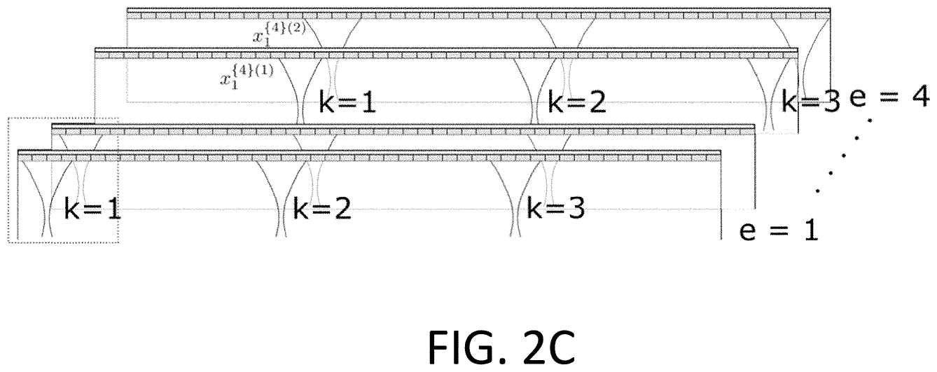

Synchronous CDMA interference is limited by the Welch Lower Bound, which describes the cross-correlation interference when several codes are transmitted in parallel. The present system and method may be used to minimize interference by focusing code transmissions to spatially separated focal zones so that clutter is minimized by both using low-interference codes and by using receive focusing to reject signals from unwanted transmit focal zones. The present system may also use code complementarity to minimize clutter along each formed A-scan line. Code complementarity will be discussed below in the context of code pairs, such as is depicted in FIG. 2a. It will be understood that, while a pair of codes is beneficial due to its simplicity, code complementarity may also be achieved using more than two codes. For example, a group of three or more codes may be found to be complementary, although doing so increases the complexity of the codes, and requires one or more additional transmit event relative to a complementary code pair as discussed herein. In addition, while it is preferred that the codes are selected to achieve complete complementarity, i.e. to generate an ideal delta function, this may not be possible or practical in all circumstances. As such, as discussed herein, complementarity will be understood to mean sufficiently complementary to minimize In the present context, complementarity refers to the sum of autocorrelations of codes in a pair adds to a delta function, as will be further discussed below. Golay codes are examples of complementary codes, however, they are not designed to be minimally interfering in an asynchronous code-division multiple access sense, and are generally restricted to binary or ternary states. The complementary pseudorandom codes proposed below may be arbitrary-level, leading to more flexibility for optimization strategies.

The present system may allow for simultaneous transmission from a larger portion of the aperture, a relative increased rate at which insonifying energy can be delivered to the imaging target, and may permit the focusing of energy to points of interest.

In general, the system and method described herein use an array of ultrasound transducers that are used to transmit “transmit events” toward the sample being imaged. Ultrasonic reflections are received from the sample, which are then processed to generate an image of the sample. This processing is based on a set of sub-image capture events received as ultrasonic reflections. Typically, the transducers both generate and receive the ultrasonic energy, although different devices may be used, if desires. In addition, the transmitter that drives the transducers, and the receiver that receives the reflections, may be part of the same electronics, which may be programmed and/or configured to perform multiple roles. Each sub-image capture event received by the receiver will be made up of a sequence of transmit events. Each transmit event involves focusing distinct waveforms on separate focal zones on the sample in a sequence, as represented by FIG. 2c. Each focal point in the sequence of transmit events will experience two or more distinct waveforms. Within each transmit event, the cross-correlation level of the distinct waveforms will be below a predetermined threshold, such as may be required to produce an image of sufficient quality for the intended purpose, and within the sequence of transmit events, the distinct waveforms that are incident on each focal zone will be complementary. As will be understood, the distinct waveforms are distinct with respect to other focal zones, in that all waveforms that are focused on a particular focal zone will be the same, but different from the waveform focused on a different focal zone. The distinct waveforms in each transmit event may be transmitted by the array, with the focusing of the waveforms being accomplished using suitable transmit delay factors for each waveform at any given transducer or row/column of transducers.

There will now be given a discussion of examples of the system and method, in which the term Parallel ULtrafast Scan-line Encoding (PULSE) is used to refer to a multiple simultaneous encoded beam framework, and the term Complementary Pseudo-Random PULSE (CPR PULSE) is used to refer to the case in which the transmitted beams are encoded using arbitrary level complementary codes. Those skilled in the art will understand that, while the discussion below is with respect to particular examples, it may be used to give context to broader concepts discussed herein.

CPR Codes

A complementary pseudorandom (CPR) code pair of length N consists of two real number sequences x⁽¹⁾, x⁽²⁾∈ [symbol] ^{N} that satisfy the complementarity condition:

Gδ=x⁽¹⁾*x⁽¹⁾+x⁽²⁾*x⁽²⁾

Here * denotes aperiodic cross correlation, G is the code gain, and δ is the delta sequence with δ₀=1 and δₜ=0 t≠0. Writing this equation in terms of code elements yields:

$$G \cdot {δ}_{j - N} = {\sum }_{i = 1}^{j} {x}_{i}^{( 1 )} {x}_{N + i - j}^{( 1 )} + {\sum }_{i = 1}^{j} {x}_{i}^{( 2 )} {x}_{N + i - j}^{( 2 )} for j = 1 , … , N .$$

For example, two codes x⁽¹⁾ and x⁽²⁾ of length N=2 form a complementary code pair when:

0=x₁⁽¹⁾x₂⁽¹⁾+x₁⁽²⁾x₂⁽²⁾

G=x₁⁽¹⁾x₁⁽¹⁾+x₂⁽¹⁾x₂⁽¹⁾+x₁⁽²⁾x₁⁽²⁾+x₂⁽²⁾x₂⁽²⁾

A graphical example of this is depicted in FIGS. 2a and 2b, Every binary complementary code is a CPR code, but there are many additional CPR codes. For example, there are only eight binary Golay codes of length two but there are an infinite number of CPR codes of length two (since there are 2N unknown CPR code values but only N restricting equations in general).

This flexibility may provide advantages. For example, consider the cₘₐₓ interference metric defined as the maximum pairwise cross correlation sum magnitude for a collection of complementary codes. For length two codes the minimum cₘₐₓ Golay interference for three codes at once is 0.5, CPR codes were found with a cₘₐₓ of 0.346, which is about a 31% reduction. Here the Welch lower bound is calculated as cₘₐₓ=0.25.

PULSE Transmission

By way of example, a model of the PULSE transmission scheme is presented, which is visualized in FIG. 1. This model helps us understand factors that produce clutter when beams are simultaneously transmitted.

To start some indexing variables are defined:

e indexes transmit event pairs, with E in total

k indexes focal zones, with K in total per event pair

p indexes transmits within a transmit pair

q indexes transducer elements, with Q in total

t indexes data vectors by time

Next, the following vector type variables are defined:

g refers to system channel data due to impulse excitation

n refers to noise

x refers to transmitted codes

y refers to received channel data

Data received by the qₜₕ element on the pₜₕ transmit within a pair of transmit events e is denoted y_{q}^{{e}(p)} and modelled as follows:

$${y}_{q}^{\{ e \} ( p )} ( t ) = {n}_{q}^{\{ e \} ( p )} ( t ) + {\sum }_{k = 1}^{K} ( {x}_{k}^{\{ e \} ( p )} * {g}_{kq}^{\{ e \}} ) ( t ) \qquad ( 1 )$$

The noise term n_{q}^{{e}(p)} is the noise vector received on the qₜₕ element on the pₜₕ transmit within a transmit event pair e. The transmitted data xₖ^{{e}(p)} is the code transmitted from sub-aperture Qₖ^{{e}} (with transmit focusing) on the pₜₕ transmit to the focal zone specified by focal zone index k and transmit event pair e (referred to as focal zone (e,k)). Finally, the impulse response channel data g_{kq}^{{e}} is the response recorded on the qₜₕ element when an impulse is transmitted with transmit focusing to focal zone (e,k) from sub-aperture Qₖ^{{e}}.

So, the data received by an element is the sum of the data associated with each simultaneous transmission, plus noise. Note that this model supports transmission of different codes to different focal zones, or on different transmit event pairs.

Location of Simultaneous Focal Zones Using PULSE

The PULSE transmission strategy can be made more general, but for the purposes of this discussion, the consideration is limited to transmission schemes with simultaneous focal zones uniformly spaced in the lateral and axial directions. The notation CPR PULSE N×M is used to refer to a CPR PULSE transmission scheme with focal zones distributed according to an N×M lateral-by-axial grid. For example, “CPR PULSE 15×3” refers to imaging 15 simultaneous lines, with 3 axial focal zones per line.

Maximum Frame Rate Acceleration Using PULSE

Traditional scan-line imaging creates an image one region at a time, forming each image region by beamforming the response from one focused beam transmitted from a sub-aperture. Therefore, the time required to form an image with scan-line imaging, T_{SL}, is the product of the total number of A-scan lines N and the time required to image one A-scan, T_{L}, so T_{S}=T_{L}N. In contrast, when using PULSE multiple beams are transmitted simultaneously, allowing several regions to be imaged in parallel. If K beams are transmitted in parallel, then the time T_{CPR} needed to form an image with CPR PULSE is T_{CPR}=(N/K)(2T_{L}). Therefore, CPR PULSE allows for an acceleration of up to K/2 relative to scan-line imaging.

Beamforming CPR PULSE Response

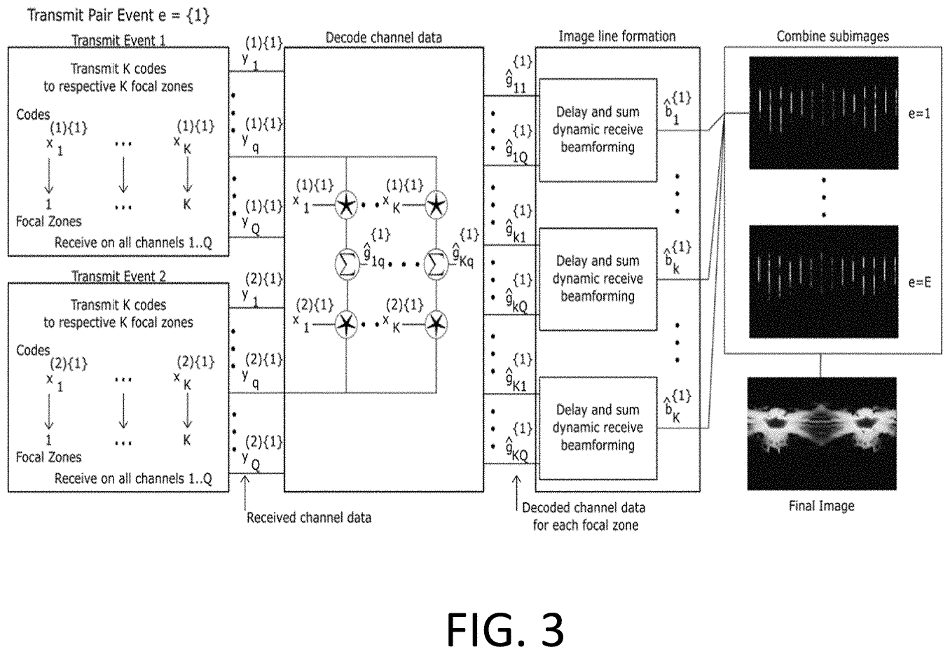

FIG. 3 illustrates the beamforming process described here. To form an image using CPR PULSE, the impulse response data ĝₖ^{{e}} is first estimated from transmit event-pair {e} associated with focal zone (e,k) and receive element q by using a matched filter of the receive data:

ĝₖ^{{e}}=xₖ^{{e}(1)}+y_{q}^{{e}(1)}+xₖ^{{e}(2)}+y_{q}^{{e}(2)} (2)

The RF-beamformed A-scan line associated with the (e,k)ₜₕ focal zone is then

$$\begin{matrix}{\hat{b}}_{k}^{\{ e \}} ( t ) = {\sum }_{q = 1}^{Q} {a}_{kq}^{\{ e \}} ( t ) {\hat{g}}_{kq}^{\{ e \}} ( t - {τ}_{q}^{k \{ e \}} ( t ) ) \\ = {\sum }_{q = 1}^{Q} {a}_{kq}^{\{ e \}} ( t ) ( {\sum }_{p = 1}^{2} {x}_{k}^{\{ e \} ( p )} ★ {y}_{q}^{\{ e \} ( p )} ) {\vert}_{t - {τ}_{q}^{k \{ e \}} ( t )}\end{matrix} \qquad ( 2 )$$

Here a is a time-dependent apodization, and τ is a dynamic time delay. Enveloping \hat{b}ₖ^{{e}} and converting from time to depth yields a single A-scan line that passes through the (e,k)ₜₕ focal zone. So, for each transmit focal zone (e,k) an A-scan line is formed by beamforming the matched filter processed channel data from pairs of complementary transmit events. Repeating this process for each A-scan line desired generates the entire image.

CPR Code Generation and Selection

CPR Code Generation

CPR code pairs may be generated using various algorithms known in the art. One example is described in: D. Egolf, T. Kaddoura and R. Zemp, “Optimization strategies and neighbour-pair complementary codes for massively parallel focal-zone ultrafast ultrasound,” 2017 IEEE International Ultrasonics Symposium (IUS), Washington, D C, 2017, pp. 1-1, which is incorporated herein by reference. This paper describes generating complementary code sets using nonlinear optimization algorithms. In another example, described below, the algorithm may be pseudorandom codes. In this example an algorithm, was seeded through pseudorandom selection of real numbers m and Aₙ. Next, the desired length of the generated codes was set as N, and a length M list S of nonnegative integers was created so that N=1+Σᵢ₌₁^{M} Sᵢ, where the first element of S is zero. Then a complementary code pair is generated with codes x⁽¹⁾ and x⁽²⁾ as follows:

xᵢ⁽¹⁾{0}=mδᵢ

xᵢ⁽²⁾{0}=0

xᵢ⁽¹⁾{n+1}=xᵢ⁽¹⁾{n}+Aₙx_{i-Sn}⁽²⁾{n}

xᵢ⁽²⁾{n+1}=Aₙxᵢ⁽¹⁾{n}+x_{i-Sn}⁽²⁾ (3)

Here {⋅} refers to the algorithm iteration, so that x⁽¹⁾{n}$ and x⁽²⁾{n} are a complementary pair of codes generated on the nₜₕ iteration. The algorithm concludes after M iterations. Subscripts refer to elements within a vector, so xᵢ⁽¹⁾{n} is the iₜₕ element of code x⁽¹⁾ on the nₜₕ iteration. Note that x_{i-Sn}^{(p)}{n}=0 is set when i−Sₙ≤0. To obtain the results in the example discussed herein, S is chosen to be a vector of ones following its first zero element, but other choices for S are also possible.

CPR Code Selection

To help select CPR codes for simultaneous transmission with low interference, the model developed above may be used to better understand the impact of code interference and focusing on image quality.

Ideally each A-scan estimate would contain little clutter associated with the transmission of several simultaneous beams. To see the impact of parallel transmission on A-scan estimation, the expression (1) is substituted for y_{q}^{{e}(p)} into the bracketed term in (2), yielding:

$${\sum }_{p = 1}^{2} {x}_{k}^{\{ e \} ( p )} {★y}_{q}^{\{ e \} ( p )} = {Gg}_{kq}^{\{ e \}} + {Γ}_{kq}^{\{ e \}} , \qquad ( 4 )$$

where Γ_{kq}^{{e}} is defined as

$${Γ}_{kq}^{\{ e \}} = {\sum }_{p = 1}^{2} ( {x}_{k}^{\{ e \} ( p )} ★ {n}_{q}^{\{ e \} ( p )} + {\sum }_{j \neq k}^{2} {x}_{k}^{\{ e \} ( p )} ★ {x}_{j}^{\{ e \} ( p )} * {g}_{jq}^{\{ e \}} )$$

and where have required the codes xₖ^{{e}(1)} and xₖ^{{e}(2)} to be complementary and have equal code gain G for all focal zones (e,k). Substituting (4) into (2) yields:

\hat{b}ₖ^{{e}}(t)=Gbₖ^{{e}}(t)+ηₖ^{{e}}(t), (5)

where ηₖ^{{e}}(t)=Σ_{q=1}^{Q}a_{kq}^{{e}}(t)Γ_{kq}^{{e}}(t−τ_{q}^{k{e}})(t)). The first term Gbₖ^{{e}}(t) is a multiple of the A-scan line formed given perfect information about the impulse response signals g_{kq}^{{e}}, which could for example be obtained by transmitting a δ-function to one focal zone at a time in a no-noise setting. The undesirable η term can be broken into two pieces ηₖ^{{e}}(t)=Nₖ^{{e}}(t)+Cₖ^{{e}}(t), where N is a noise term and C represents clutter from other focal zones. The noise term is given by:

$${N}_{k}^{\{ e \}} ( t ) = {\sum }_{q = 1}^{Q} {a}_{kq}^{\{ e \}} ( t ) ( {\sum }_{p = 1}^{2} {x}_{k}^{\{ e \} ( p )} * {n}_{q}^{\{ e \} ( p )} ) {\vert}_{t - {τ}_{q}^{k \{ e \}} ( t )}$$

and the term representing clutter from other focal zones is given by:

$${C}_{k}^{\{ e \}} ( t ) = {\sum }_{q = 1}^{Q} {a}_{kq}^{\{ e \}} ( t ) ( {\sum }_{j \neq k}^{2} {c}_{kj}^{\{ e \}} * {g}_{jq}^{\{ e \}} ) {\vert}_{t - {τ}_{q}^{k \{ e \}} ( t )} \\ with {c}_{kj}^{\{ e \}} = {\sum }_{p = 1}^{2} {x}_{k}^{\{ e \} ( p )} ★ {x}_{j}^{\{ e \} ( p )}$$

To maximize quality of reconstruction, ηₖ is minimized. Cₖ^{{e}} represents the undesirable clutter associated with transmitting on multiple focal zones simultaneously.

This result implies that reducing the magnitude of the beamformed line crosstalk g_{jq}^{{e}} with j≠k will tend to reduce the clutter introduced around focal zone (k,e), which can be accomplished by increasing the spacing between simultaneous beams or by transmitting these beams in different directions.

In addition, it can be seen that reducing the cross correlation sum cₖⱼ^{{e}} of codes associated with different focal zones will tend to reduce clutter. Therefore, it seems plausible the total clutter associated with simultaneous focal zones (e,k) and (e,j) will increase with the integrated side lobe level metric ISLₖⱼ=Σₜ(cₖⱼ^{{e}}(t))² for k≠j.

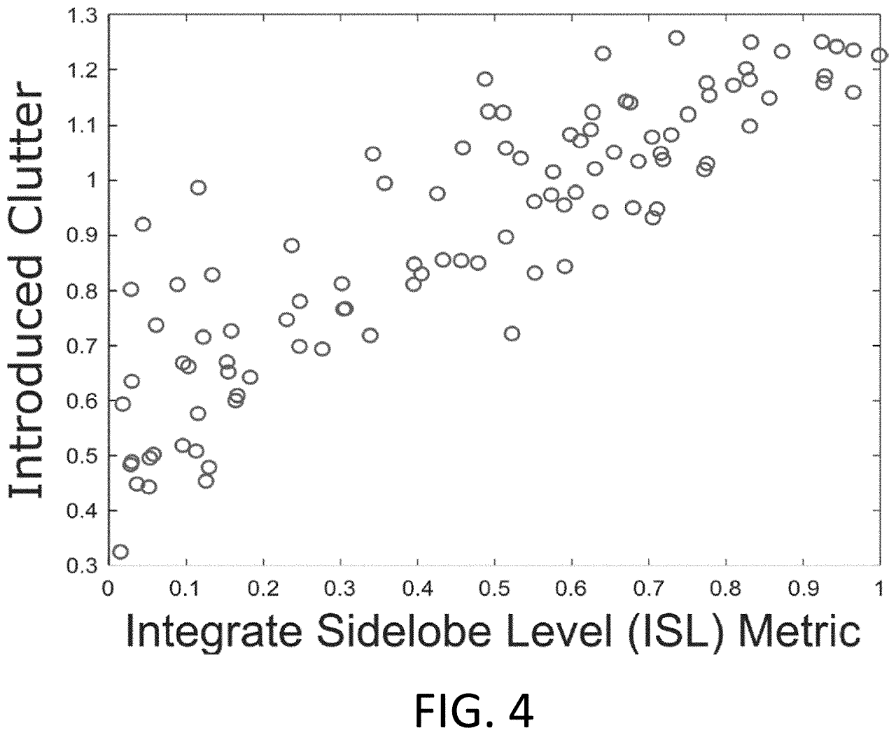

Indeed, upon simulating simultaneous transmission of pairs of beams with a variety of encoding schemes, it was observed that ISLₖⱼ was correlated to the introduced clutter, as shown in FIG. 4, which depicts the increase in clutter introduced by simultaneous transmission with the ISL metric. Each point corresponds to the ISL metric of a pair of simultaneously transmitted codes, along with the resulting introduced clutter. Clutter was defined as the square of the sum of the error image values: clutter=ΣₓΣ_{z}(Ref(x,z)−I(x,z))², where Ref is a reference image formed using CPR PULSE with a single beam, and I is the image obtained using two beams at once. The phantom used consisted of two point scatterers. The advantage of the ISL metric is that it is object independent and depends only on the properties of the codes. In contrast, the clutter metric is dependent on the object imaged. FIG. 4 illustrates that ISL may be used as a valuable metric for picking code pairs and may be highly correlated with image quality.

To pick the codes used for simulation and experimental testing, 1000 code sets were generated, and those that minimized the ISL metric were picked for the simultaneously transmitted beams. A code length of 10 was chosen, as it offered reasonable design flexibility as well as an acceptably small dead-zone (the initial depth where no useful image can be formed owing to amplifier saturation due to transmission).

CPR PULSE Implementation

Simulation

Simulation were conducted using a 5 MHz center frequency linear array transducer with 128 elements, a kerf of 20 μm, an element width of 200 μm, an element height of 5 mm, and total width of 3.94 cm. Simulation sampling frequency was 100 MHz, and beamforming was performed using a beamforming toolbox. Hanning apodization was used on receive sub-apertures except as noted.

To show CPR PULSE feasibility in the static case, simulations were conducted using a grid of point scatterers and a cyst phantom. In those simulations, the grid consisted of nine evenly spaced point scatterers distributed across 12 mm axially and 10 mm laterally. The cyst phantom used a total of 75,000 scatters, and contained nine anechoic circular regions of varying radius equally spaced in a three by three grid. These cysts were surrounded by a large number of additional scatterers with scattering strength given by a Gaussian distribution. This large number of scatterers more closely approximates the scattering of human tissue than the small number of scatterers used in the grid of point scatterers.

To show CPR PULSE feasibility in a context with motion, an axially moving grid was simulated of nine points distributed across 10 mm axially and 25 mm laterally. The grid of points was first set to move at 1 m/s and imaged at 837 frames per second (fps) using (1) coherent plane wave compounding with 16 transmits, and (2) 16×3 CPR PULSE with 16 transmits. The grid of points was then set to move at 4 m/s and imaged at 1673 fps using (1) coherent plane wave compounding with 8 transmits, and (2) 32×3 CPR PULSE with 8 transmits.

Experiment

The simulation was implemented using a programmable ultrasound system (Vantage 256, Verasonics, US) with a 5 MHz 128-element imaging transducer array (L7-4, Philips ATL, WA). This system uses tri-state pulsers as opposed to arbitrary function generators, requiring conversion of the arbitrary level codes into tri-state form for transmission. This was achieved using pulse width modulation and the Verasonics Vantage Arbitrary Waveform Toolbox. However, the conversion process requires the codes to lie in the transducer bandwidth. To bandwidth match, each code value was repeated 25 times, the number of repetitions necessary to match code autocorrelation peak width to the period associated with transducer center frequency. After repeating code elements, each code was convolved with the electromechanical impulse response of the L7-4 transducer (experimentally measured with a hydrophone submerged in water). After code value repetition the resulting codes had a final length of 250 samples, implying a dead-zone of 0.77 μm in water on the 250 MHz sampling frequency system.

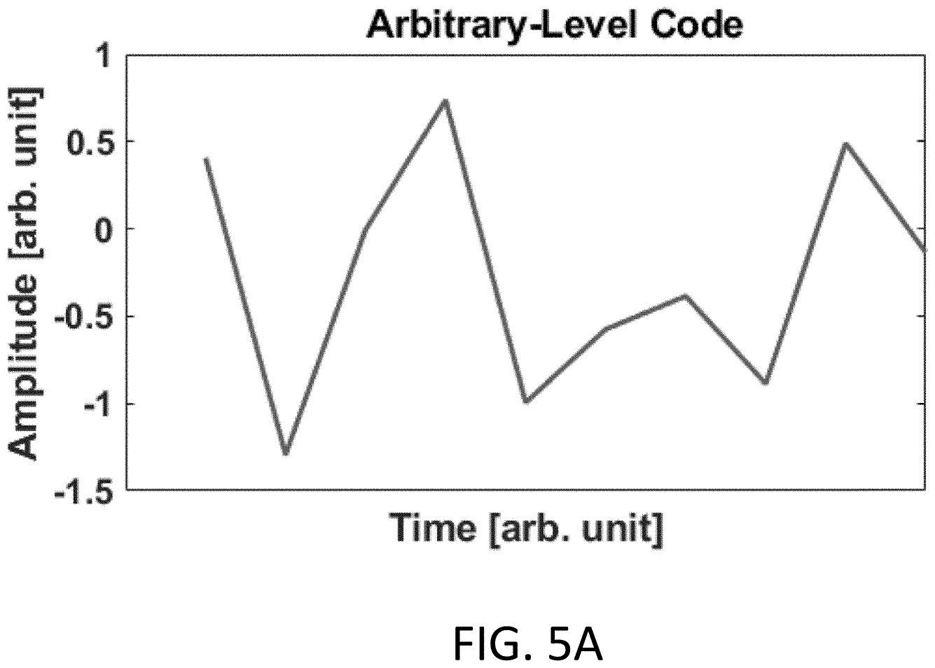

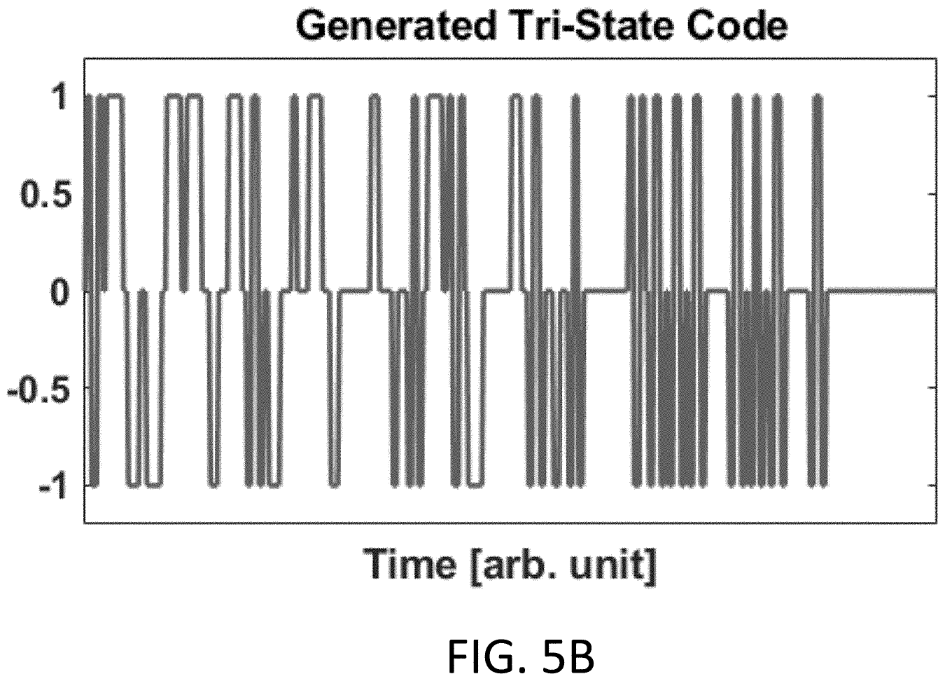

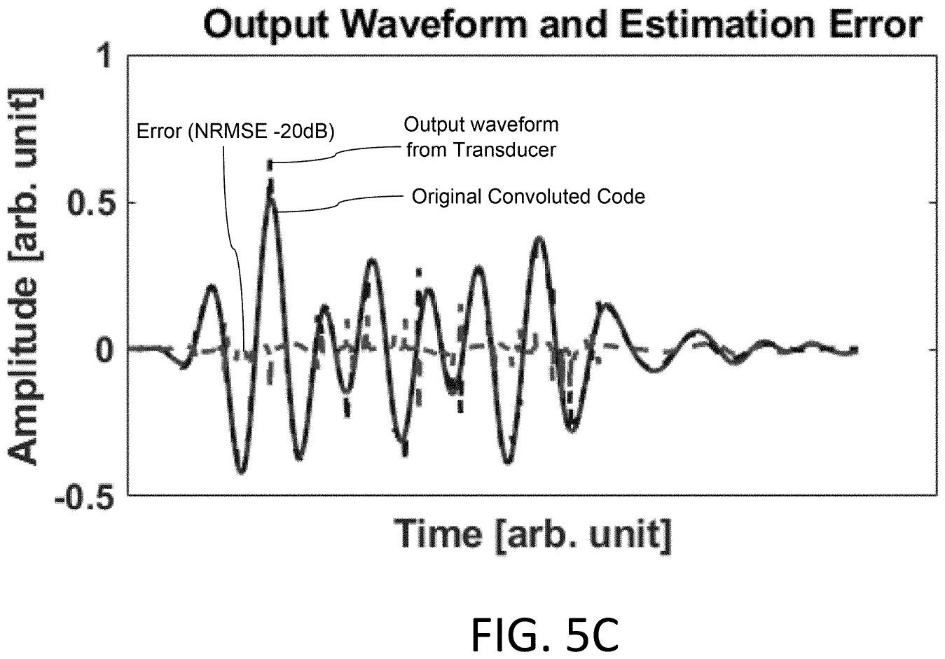

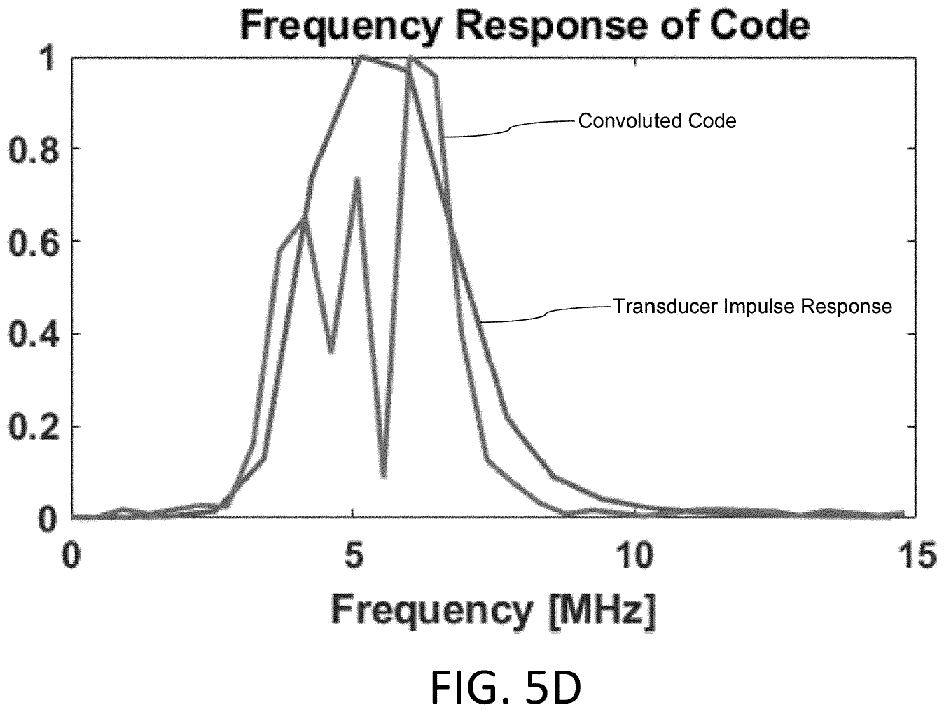

FIG. 5a-5d illustrate the process used for the experimental transmission of a CPR code, as shown in FIG. 5a. The code is first converted to tri-state form as shown in FIG. 5b and then convolved with the transducer impulse response to yield the solid line shown in FIG. 5c. An experimental measurement of the final transmitted code, together with the error associated with implementation is also shown in FIG. 5c. The normalized root-mean-square error in conversion was −20 dB. FIG. 5d illustrates the efficacy of the bandwidth matching process. In fact, it was found that the percentage of power within the 2-8 MHz bandwidth to improve from 29% to 70% with code element repetition.

The experimental phantom used was the tissue-mimicking ATS-539 phantom (ATS Laboratories, CT, USA), used commercially for ultrasound imaging system quality assurance. This phantom has an attenuation coefficient of 0.5 dB/cm/MHz, similar to that of human tissue.

To show the feasibility of experimental implementation of CPR PULSE for a range of simultaneous focal zones, the cyst phantom was imaged using CPR PULSE 3×3, 7×3, and 15×3. Transmit subaperture size was set to 64 elements, implying F-numbers of 1.57, 2.10, and 2.62 at focal depths of 30 mm, 40 mm, and 50 mm. Image reconstruction was performed using dynamic receive beamforming with a constant F-number of 1.05. A baseline was established for acceptable image quality by also imaging using coherent plane wave compounding at the same frame rate as the CPR PULSE implementations tested (implying 85, 36, and 17 angled transmissions). For simplicity, the maximum voltage values (20 V) used by the CPR PULSE and plane wave implementations was matched.

To show CPR PULSE feasibility with respect to safety, biosafety measures described by the ODS (Optical Display Standard) were determined and compared those to FDA standards for ultrasound safety limitations. For each CPR PULSE configuration, pressure measurements were obtained with a calibrated hydrophone (ONDA HNP—0400) submerged in water. The spatial peak of the ultrasound field was first located with the hydrophone by scanning the ultrasound field laterally and axially. The hydrophone was then held stationary at the spatial peak while transmitting the CPR PULSE configuration under test, where it recorded the pressure-time tracing for 1 s of imaging. A peak voltage level of 20 V was used for all safety tests.

Results

Static Simulation

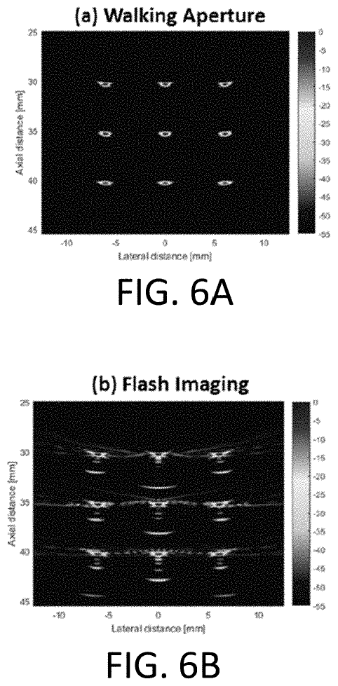

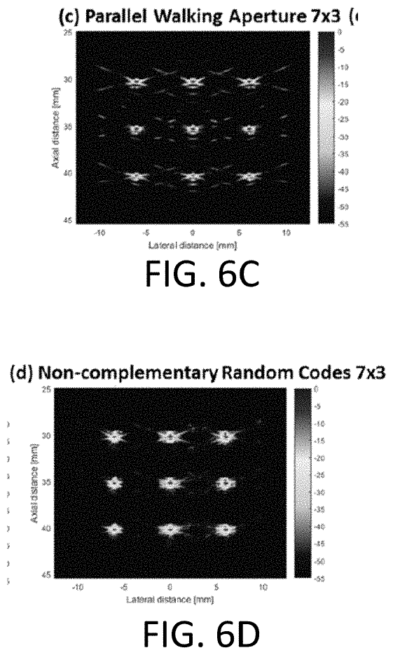

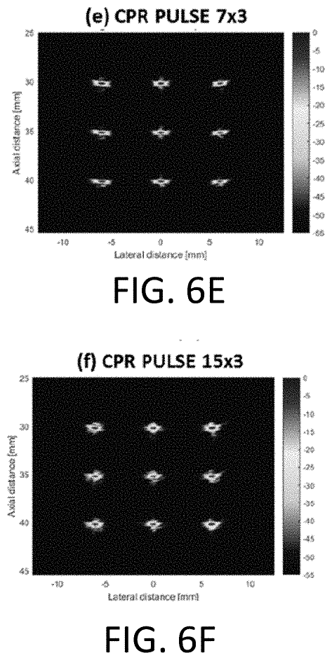

FIGS. 6e and 6f show the performance of CPR PULSE, FIG. 6b shows plane wave imaging, FIG. 6a shows a traditional walking aperture in simulation when imaging a 3×3 grid of point scatterers. FIG. 6c is a comparison of the transmission of a single-cycle 5 MHz sine pulse to multiple simultaneous focal zones as well as transmission of non-complementary pseudo-random codes FIG. 6d.

It may be observed that CPR PULSE obtains acceptable images, with increasing degradation present as the number of simultaneous focal zones is increased. This degradation occurs in the form of reduced lateral and axial resolution point spread functions (PSFs), as well as lower intensity distributed clutter. If non-complementary codes are used, it was observed that even greater PSF degradation and additional distributed clutter.

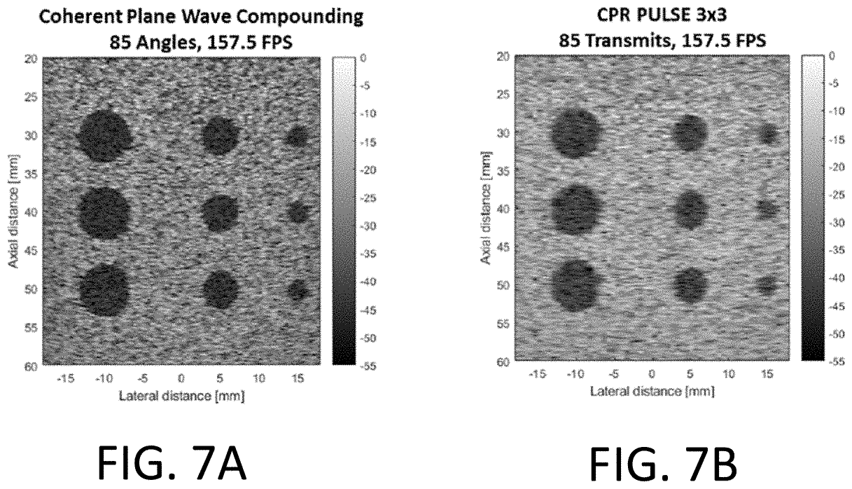

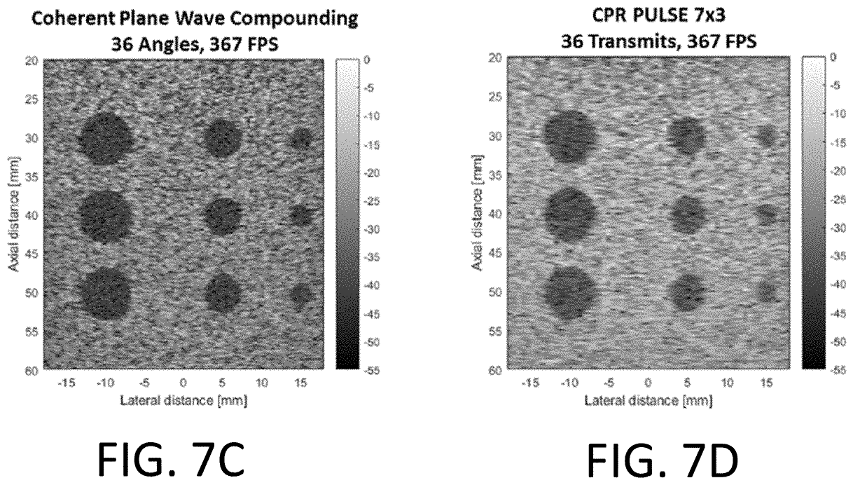

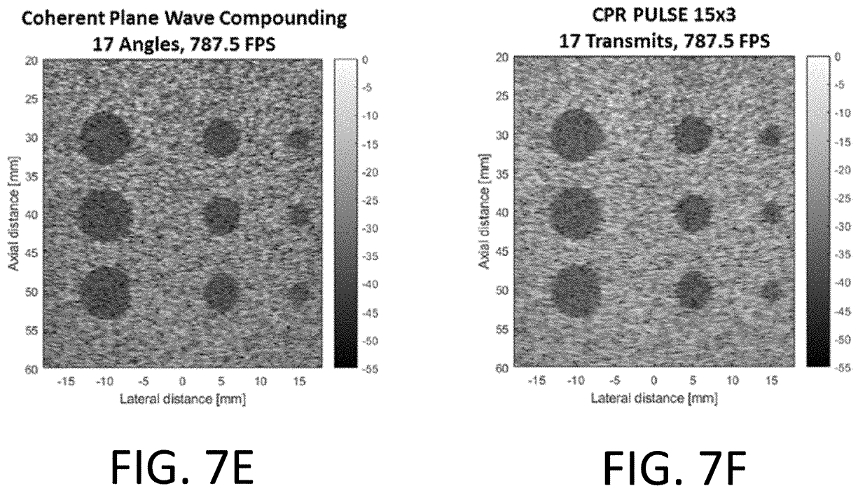

FIG. 7a shows the results of cyst phantom simulation for coherently compounded plane-waves with 85 angles, FIG. 7b shows results for coherently compounded plane-waves with 36 angles, FIG. 7c shows results for coherently compounded plane-waves with 17 angles, FIG. 7d shows results for CPR PULSE 3×3 with 9 focal zones, FIG. 7e shows results for CPR PULSE 7×3 with 21 focal, and FIG. 7f shows results for CPR PULSE 15×3 with 45 focal zones.

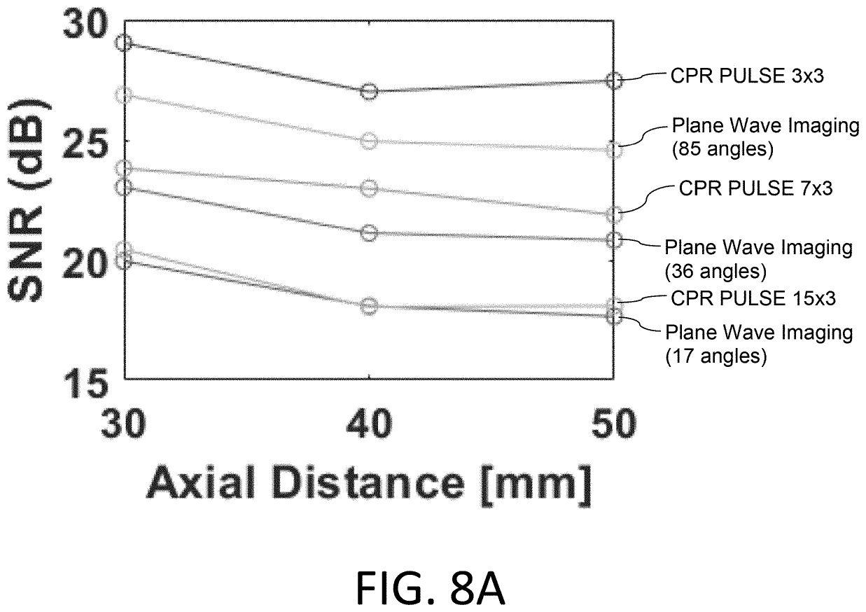

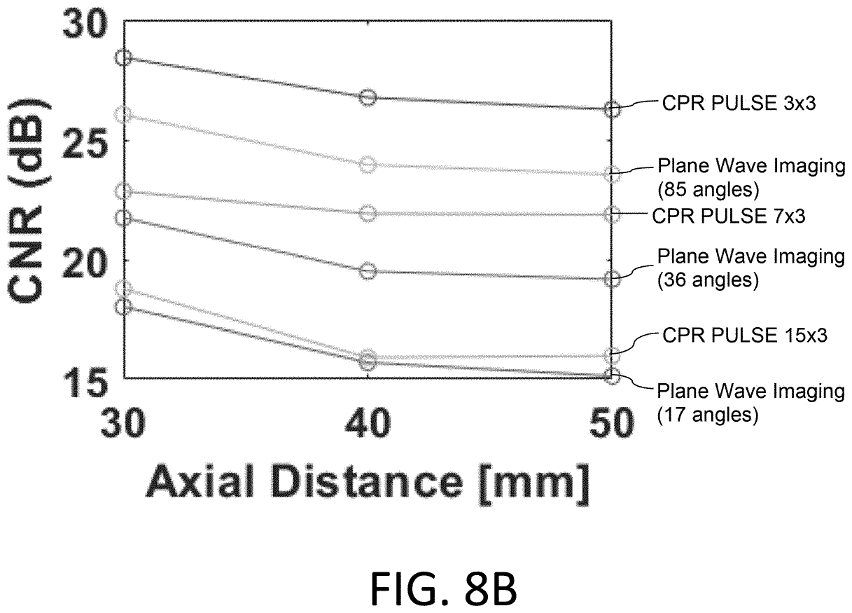

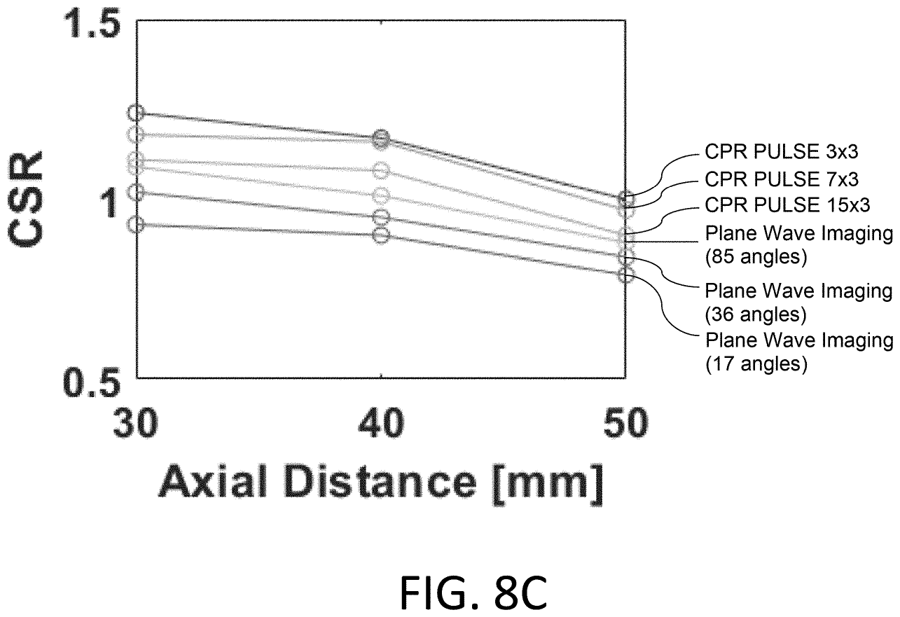

To quantify contrast-lesion detection capability when imaging the cyst phantom, contrast-to-speckle ratio (CSR), contrast-to-noise ratio (CNR), and signal-to-noise ratio (SNR) can be calculated for the cyst targets. These may be defined respectively as |Sᵢₙ−S_{bg}|/\sqrt{σᵢₙ²+σ_{bg}²}, 20 log₁₀(|Sᵢₙ−S_{bg}|/σₙ), and 20 log₁₀(S_{bg}/σₙ). Here, S refers to mean signal, σ refers to standard deviation, in refers to a cyst interior, bg refers to the background, and n refers to noise. These metrics may be calculated for each cyst target in an image, and then calculate an average metric by averaging metric values for all cysts at a given depth, with results for SNR shown in FIG. 8a, CNR shown in FIG. 8b, and CSR shown in FIG. 8c.

In the cyst simulation context, CPR PULSE obtained performance comparable to plane wave compounding both qualitatively and quantitatively. Note that cyst visibility is reduced as the number of simultaneous CPR focal zones is increased or as the number of compounded plane waves is reduced, agreeing with the general trends seen for the simulation of a grid of point scatterers.

Simulation with Motion

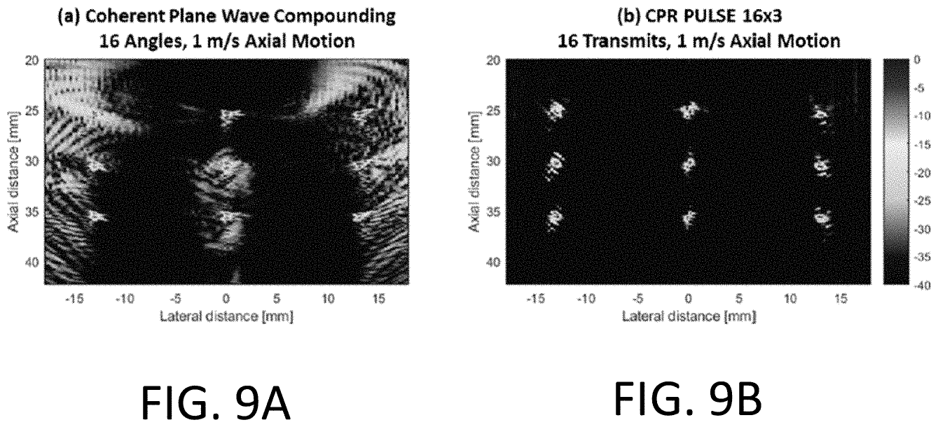

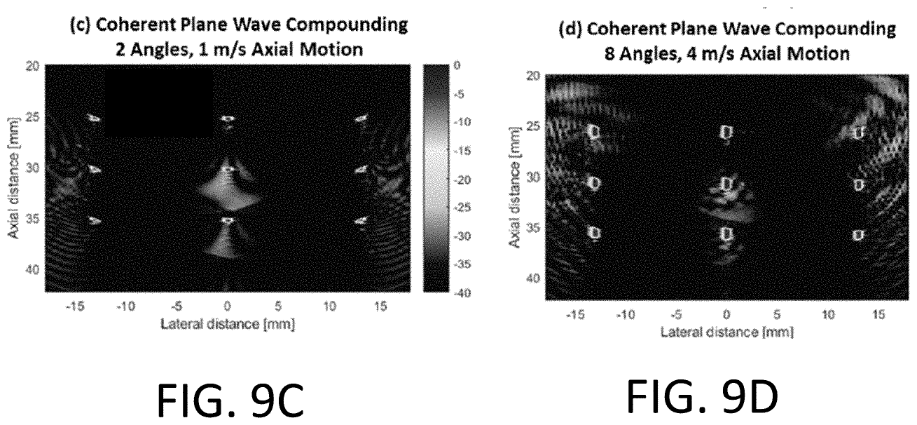

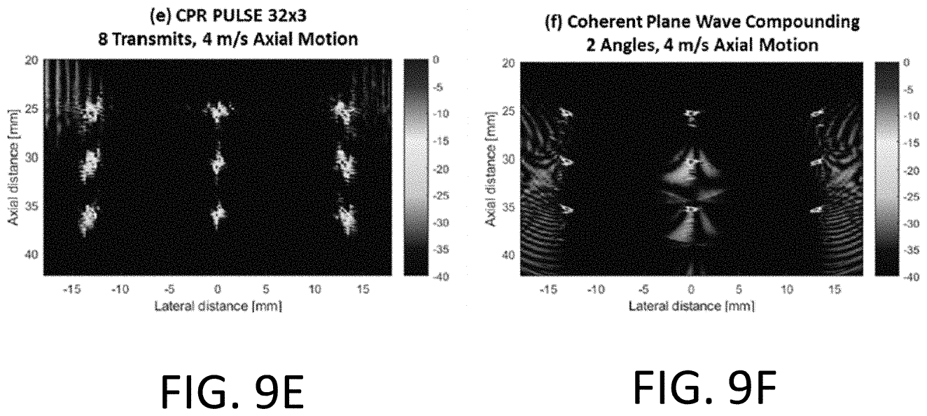

Additional advantages of the CPR approach described herein may also be achieved when motion is present. FIG. 9a shows simulated images acquired of an axially moving grid of nine points with a velocity of 1 m/s and 16 angle coherent plane wave compounding, FIG. 9b with a velocity of 1 m/s 16×3 CPR PULSE, FIG. 9c with a velocity of 1 m/s and 2 angle coherent plane wave compounding, FIG. 9d with a velocity of 4 m/s and 8 angle coherent plane wave compounding, FIG. 9e with a velocity of 4 m/s and 32×3 CPR PULSE, and FIG. 9f with a velocity of 4 m/s and 2 angle coherent plane wave compounding. An imaging frame rate of 837 fps was used in FIGS. 9a and 8b, 1673 fps in FIG. 9d-9e, and 6692 fps in FIGS. 9c and 9f. It was observed that CPR PULSE enjoyed reduced motion artifacts at both movement velocities.

Experimental Cyst Phantoms

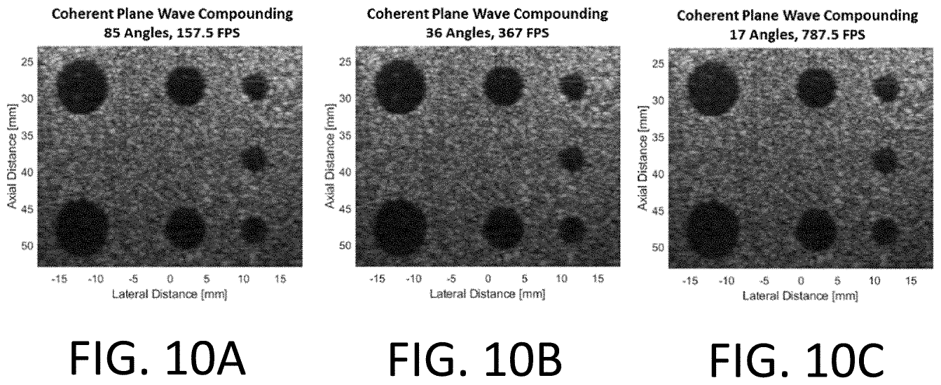

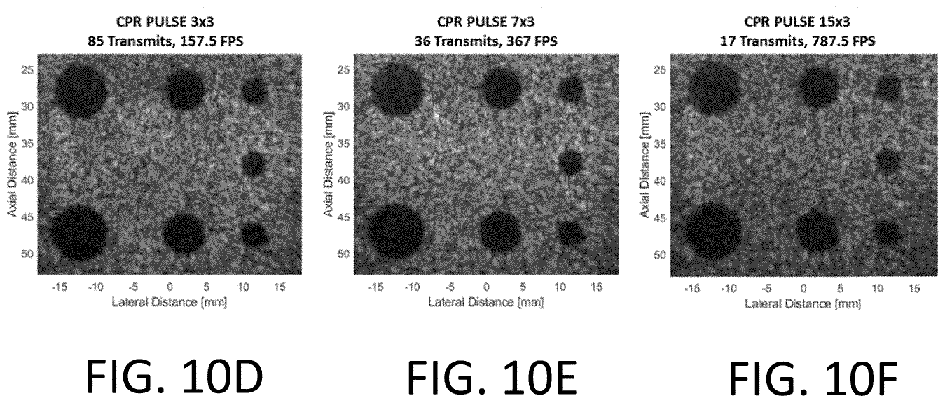

FIG. 10a compares experimentally obtained images of the ATS-539 phantom with anechoic regions for coherently compounded plane-waves with 85 angles, FIG. 10b for coherently compounded plane-waves with 36 angles, FIG. 10c for coherently compounded plane-waves with 17 angles, FIG. 10d for CPR PULSE with 9 focal zones equally spaced in a 3×3 grid, FIG. 10e for CPR PULSE with 21 focal zones in a 7×3 grid, and FIG. 10f for CPR PULSE with 45 focal zones in a 15×3 grid.

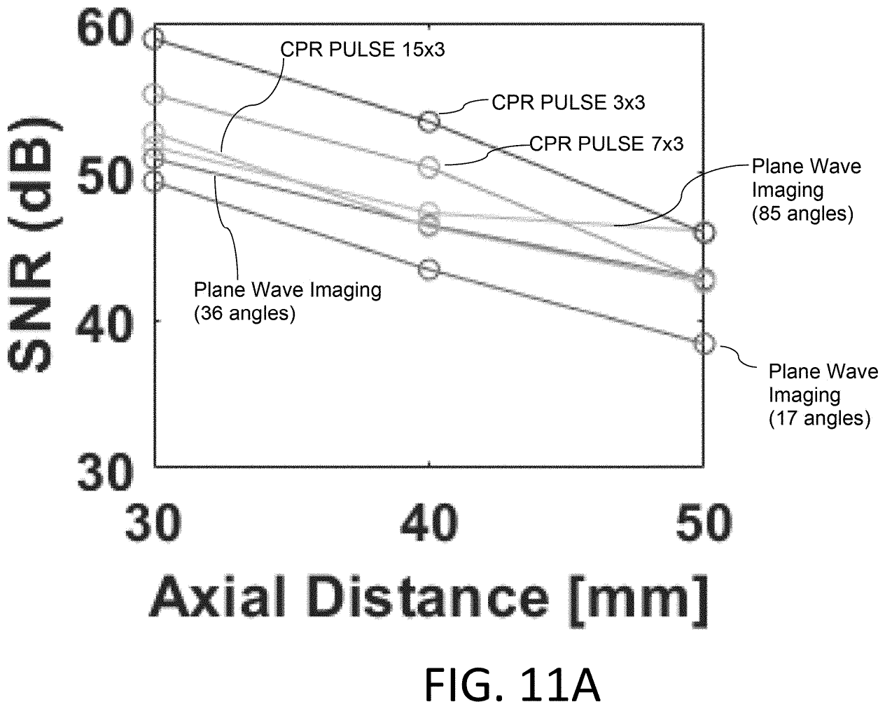

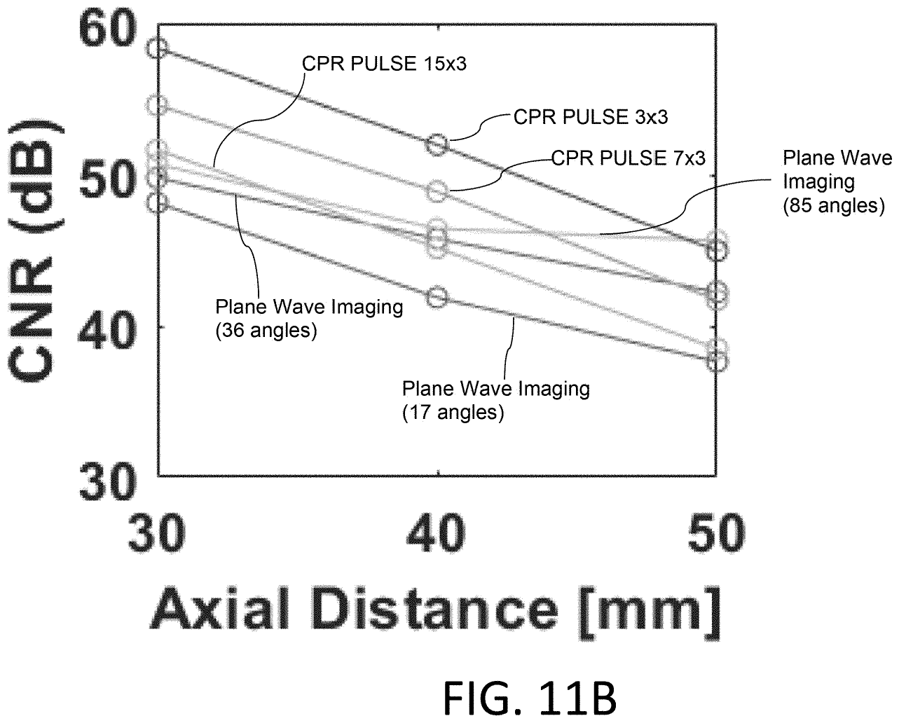

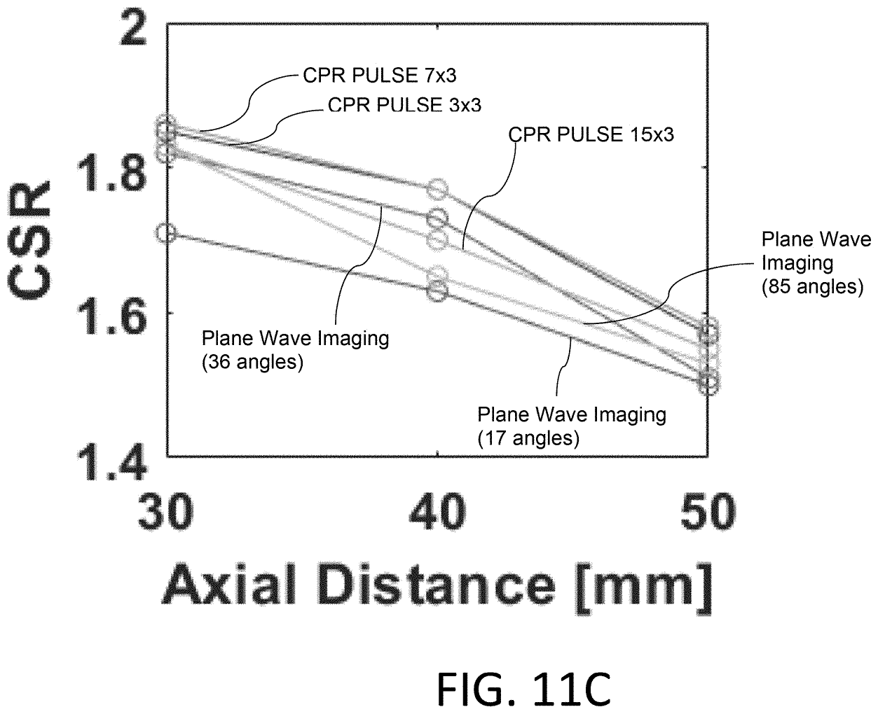

Cyst imaging performance of CPR PULSE and coherently compounded plane wave imaging was quantified using the metrics defined above, including the SNR metrics as shown in FIG. 11a, CNR metrics as shown in FIG. 11b, and CSR metrics as shown in FIG. 11c. As can be seen, SNR, CNR, and CSR decreased with depth across all imaging methods. As in simulation, it was observed that performance degrades as the number of simultaneous CPR PULSE focal zones is increased or the number of plane waves compounded is decreased,

The axial and lateral resolution of the CPR PULSE imaging scheme was measured with the same tissue-mimicking ATS-539 phantom. An axial resolution of 240 μm, and a lateral resolution of 520 μm was calculated. By comparison, plane wave imaging obtained an axial resolution of 150 μm, and a lateral resolution of 410 μm.

Safety

TABLE 1
Safety metric measurements of CPR PULSE imaging scheme

| | 3 × 3 | 7 × 3 | 15 × 3 |
|---|---|---|---|
| Iₛₚₜₐ (mW/cm²) | 150 | 210 | 170 |
| MI | 0.97 | 0.71 | 0.57 |
| TIS/TIB | 0.81 | 0.76 | 0.70 |
| (soft tissue at surface) | | | |
| TIS/TIB | 0.71 | 0.81 | 0.73 |
| (scanned large aperture) | | | |

Pressure measurements obtained were first de-rated by 0.3 dB/cm-MHz as described in the ODS, and then used to calculate the Iₛₚₜₐ (Intensity spatial-peak-temporal-average), Mechanical Index (MI), and Thermal Index (TI) for each imaging method.

The ultrasound safety standard describes multiple tissue models that estimate TI for scanned and unscanned modalities. Note that the modality used herein is best described as either a scanned or unscanned modality depending on the number of simultaneous focal zones. In this example, the maximum number of beams transmitted in parallel was 15, spaced across the full aperture of the array. For this reason, the Thermal Index for Soft Tissue (TIS), and the Thermal Index for Bone (TIB) for the scanned, large-aperture case was calculated. Metric values were also calculated for the general soft-tissue-at-surface model as described in the ODS.

The results for all safety measures are summarized in Table I. It was observed that the MI decreases as the number of simultaneous beams used for imaging is increased. All CPR PULSE configurations tested (using a peak of 20 V) did not exceed safety limits.

### DISCUSSION

The example described herein demonstrates that the feasibility of implementing the CPR PULSE imaging scheme on a programmable research ultrasound platform. As a key part of this feasibility demonstration, it has been shown that it is possible to implement arbitrary level codes (such as CPR codes) on a tri-state pulser with an error of only −20 dB. Feasibility was demonstrated in simulation and experiment, where images comparable to those obtainable with more standard techniques were acquired while using arbitrary level coded excitation and highly parallel focal schemes (including schemes with axial stacking of focal zones). The implemented scheme extends both multi-line transmission schemes and Golay imaging schemes.

It should be noted that imaging comparisons were performed using the same maximum voltage for simplicity. Currently, the CPR approach described herein has safety metrics well below those permitted by ANSI and may be limited by the system and associated pulser limitations. Future system improvements may offer significant improvements in SNR and imaging depth. Future work should compare plane-wave approaches when matching various safety metrics for various numbers of transmits.

Interestingly, it was observed that the mechanical index metric decreased as the number of simultaneous beams was increased. This may be because each total composite transmission excitation was constructed by summing the excitation required for each beam individually, and then normalizing the result to its maximum. This approach results in high intensities at the overlap of adjacent beams, which (together with the normalization applied) results in lower intensities being transmitted from most of the transducer array. Consequently, power of transmission decreases with the number of simultaneous beams.

Like other ultrafast ultrasound methods, the number of frames that can be acquired at these high frame rates may be limited by the system memory. On the programmable ultrasound system used in the example described above, the size of the matrix required to hold all the RF Data to construct one imaging frame for the 15×3 focal zone case is 9 MiB. Given a system memory of 32 GiBs and a given size of image, the system may hold, for example, about 3640 frames. Imaging at 787.5 FPS, this translates to 4.6 seconds of data acquisition.

The fact that CPR PULSE is feasible to implement is interesting because it opens up a very large design space. As noted above, Golay codes form a small subset of CPR codes, and so there is a great deal of code optimization to be explored. For example, CPR codes could be optimized to further reduce inter-beam interference, or to increase motion robustness.

In addition to the example described above, a larger number of focal zones in patterns may be strategically packed, other than in a laterally-linear or radial spread. This sort of imaging scheme takes further advantage of the increased flexibility with respect to directivity of imaging energy afforded by focused imaging strategies. Applications of this motif may also be used in phased array or 2D array contexts.

As expected for a multi-line transmission strategy, it was observed that in the presence of both 1 m/s and 4 m/s motion, CPR PULSE obtained images with decreased clutter in the presence of axial motion compared to those obtained by coherently compounded plane waves. This may be because CPR PULSE only interrogates each spatial location twice, while plane wave compounding interrogates each spatial location on each transmission. Consequently, in the case of motion, the beamforming process in the plane wave compounding case may have to incorporate information from a greater spread of scatterer locations than CPR PULSE does.

For plane-wave approaches to be robust to motion, fewer transmits may be required. However, when only two transmits are used, the image quality may be degraded whether the target is moving or not. Given that the CPR PULSE approach requires coherency over only two transmits, robustness to motion may be a key advantage.

Experimentally, imaging frame-rates up to 787.5 frames per second have been demonstrated with minimal image degradation. Future work will aim to assess the performance of this approach in cardiac imaging and other applications where significant tissue motion is present and where high-frame-rates will better capture cardiovascular dynamics and potentially lead to visualization of flow in the coronary arteries.

The CPR approach currently offers slightly degraded experimental resolution compared to plane-wave approaches but may be improved in the future given that simulations provided effectively non-degraded resolution results. Simulated signals account for transducer electromechanical response but do not pre-convolve the codes with the response as was done in experiments. The pre-convolution of codes in experiments was necessary owing to limitations of the tri-state pulser and bandwidth limitations. In the future, an arbitrary-level pulser may enable code transmission without pre-convolution, thus improving resolution.

In this patent document, the word “comprising” is used in its non-limiting sense to mean that items following the word are included, but items not specifically mentioned are not excluded. A reference to an element by the indefinite article “a” does not exclude the possibility that more than one of the elements is present, unless the context clearly requires that there be one and only one of the elements.

The scope of the following claims should not be limited by the preferred embodiments set forth in the examples above and in the drawings, but should be given the broadest interpretation consistent with the description as a whole.

What is claimed is:

1. An ultrasound imaging system for imaging a sample, comprising:
an array of ultrasound transducers;
a transmitter for driving the array of ultrasound transducers;
a receiver that receives ultrasonic reflections from the sample;
a processor that generates an image of the sample based on a set of sub-image capture events, each sub-image capture event comprising received ultrasonic reflections; and
a controller comprising instructions to, for each sub-image capture event, cause the transmitter to transmit a sequence of transmit events from the ultrasound transducers, each transmit event comprising a plurality of distinct waveforms directed toward separate focal zones on the sample, and the sequence of transmit events comprising a sequence of distinct waveforms directed toward each focal zone, wherein a cross-correlation level of the distinct waveforms in each transmit event is below a predetermined threshold, and wherein each sequence of distinct waveforms directed toward each focal zone are complementary.

2. The ultrasound imaging system of claim 1, wherein the predetermined threshold is selected to produce a desired image quality.

3. The ultrasound imaging system of claim 1, wherein the sequence of distinct waveforms are generated using nonlinear optimization algorithms.

4. The ultrasound imaging system of claim 1, wherein the sequence of distinct waveforms are pseudorandom codes or Golay codes.

5. The ultrasound imaging system of claim 1, wherein the plurality of distinct waveforms in each transmit event are transmitted simultaneously, and are directed toward separate focal zone using transmit delays across the array of ultrasound transducers.

6. The ultrasound imaging system of claim 1, wherein the complementarity of the sequence of distinct waveforms is such that a sum of an aperiodic autocorrelation of the sequence of distinct waveforms approximates a discrete delta function.

7. A method of ultrasound imaging of a sample, comprising the steps of:
driving an array of ultrasound transducers to transmit events toward the sample;
receiving ultrasonic reflections from the sample as a set of sub-image capture events, each sub-image capture event comprising a sequence of transmit events; and
generating an image of the sample based on the set of sub-image capture events;
wherein each transmit event comprises a plurality of distinct waveforms directed toward separate focal zones on the sample, and each sequence of transmit events comprising a sequence of distinct waveforms directed toward each focal zone, wherein a cross-correlation level of the distinct waveforms in each transmit event is below a predetermined threshold, and wherein each sequence of distinct waveforms directed toward each focal zone are complementary.

8. The method of claim 7, wherein the predetermined threshold is selected to produce a desired image quality.

9. The method of claim 7, further comprising the step of generating the distinct waveforms using nonlinear optimization algorithms.

10. The method of claim 7, wherein the sequence of distinct waveforms are pseudorandom codes or Golay codes.

11. The method of claim 7, wherein the plurality of distinct waveforms in each transmit event are transmitted simultaneously, and are directed toward separate focal zone using transmit delays across the array of ultrasound transducers.

12. The method of claim 7, wherein the complementarity of the sequence of distinct waveforms are such that a sum of an aperiodic autocorrelation of the sequence of distinct waveforms approximates a discrete delta function.
